(19) United States
(12) Patent Application Publication
(10) Pub. No.: US 2023/0108369 A1
(43) Pub. Date: Apr. 6, 2023

(54) ENHANCED SYSTEMS AND PROCESSES FOR BLOCKCHAIN TOKENS AND LEDGER FOR HEALTHCARE TRANSACTIONS

(71) Applicant: Farmgate Media LLC, Katy, TX (US)

(72) Inventor: Alex Bachoura, College Station, TX (US)

(21) Appl. No.: 17/495,627

(22) Filed: Oct. 6, 2021

(51) Int. Cl. G06Q 20/36; G06Q 20/10; G06Q 20/38; G16H 10/60

(52) U.S. Cl. CPC G06Q 20/3678; G06Q 20/102; G06Q 20/389; G16H 10/60

(57) ABSTRACT

Devices, systems, and methods are provided for blockchain tokens and ledger for medical and dental transactions. A method may include determining a cost of a healthcare service provided to a patient by a healthcare provider; accessing a blockchain digital ledger; identifying a digital wallet of the patient stored using the blockchain digital ledger; identifying, a digital token stored in the digital wallet, the digital token restricted to payment for the healthcare service and issued by an insurer; transferring, based on a smart contract of the blockchain digital ledger, the digital token from the first digital wallet of the patient to the healthcare provider; determining a difference between the cost and a pecuniary amount provided by the digital token; and generating a bill for the patient, the bill indicating that the patient owes the difference to the healthcare provider.

### TECHNICAL FIELD

This disclosure relates to systems, methods, and devices for enhanced blockchain tokens and ledgers for healthcare transactions.

### BACKGROUND

Healthcare and dental providers may lack access to insurance benefits of a patient and may need to coordinate with insurance providers to determine the amount of money owed by a patient. A healthcare or dentistry provider's process for determining insurance coverage and the amount owed by a patient can be lengthy and inefficient.

### BRIEF DESCRIPTION OF THE DRAWINGS

FIG. 1 illustrates an example system for a blockchain ledger available to healthcare providers and patients, in accordance with one or more example embodiments of the present disclosure.

FIG. 2 illustrates an example system flow for using a blockchain ledger available to healthcare providers and patients, in accordance with one or more example embodiments of the present disclosure.

FIG. 3 illustrates an example system for a blockchain ledger and non-fungible tokens available to healthcare providers and patients, in accordance with one or more example embodiments of the present disclosure.

FIG. 4 illustrates an example process flow for using a blockchain ledger available to healthcare providers and patients, in accordance with one or more example embodiments of the present disclosure.

FIG. 5 is a block diagram illustrating an example of a computing device or computer system upon which any of one or more techniques (e.g., methods) may be performed, in accordance with one or more example embodiments of the present disclosure.

Certain implementations will now be described more fully below with reference to the accompanying drawings, in which various implementations and/or aspects are shown. However, various aspects may be implemented in many different forms and should not be construed as limited to the implementations set forth herein; rather, these implementations are provided so that this disclosure will be thorough and complete, and will fully convey the scope of the disclosure to those skilled in the art. Like numbers in the figures refer to like elements throughout. Hence, if a feature is used across several drawings, the number used to identify the feature in the drawing where the feature first appeared will be used in later drawings.

The following description and the drawings sufficiently illustrate specific embodiments to enable those skilled in the art to practice them. Other embodiments may incorporate structural, logical, electrical, process, algorithm, and other changes. Portions and features of some embodiments may be included in, or substituted for, those of other embodiments. Embodiments set forth in the claims encompass all available equivalents of those claims.

### DETAILED DESCRIPTION

Blockchain computing systems enable healthcare patients and their caregivers (“patients”), healthcare service providers such as doctors, nurses, nurse practitioners, dentists, clinics, and hospitals (“service providers”), and non-healthcare entities like insurance companies, clinical research organizations, and healthcare product merchants “third parties” to communicate with each other and conduct secure, efficient, privacy compliant transactions. Healthcare entities can include providers or organizations providing various services and goods, including medical, dental, chiropractic, herbal medicine, psychology, or others. The use of smart contracts published to the blockchain ledger associated with the blockchain computing system provides trustworthy and efficient interactions between these healthcare industry participants. Some current blockchain-implemented healthcare systems may struggle to prevent duplication of individual patient data, especially about patient healthcare records. Some current Off-Chain Computer Systems may not provide for conversion of records, scans, and diagnosis into healthcare records. Providers in some current blockchain implemented systems may not have access to a patient's digital wallet, decreasing efficiency in billing as the providers rely on insurance companies to provide insurance benefits information. In addition, the conversion of data to a format that is readily usable, but still secure, on the blockchain is an issue in some techniques and systems.

In particular, some healthcare systems require a provider to contact or remain in contact with third parties like insurance companies to confirm insurance benefits, payment amounts owed by patients, and receive payments for the value of the services or goods provided. For example, a patient's insurance may cover at least a portion of a procedure or product. To determine what, if any, portion of a procedure or product is covered (e.g., paid for) by an insurer, a healthcare provider may submit to the patient's insurer a claim that details the services and products provided to the patient, and their costs. In response, the insurer may generate an Explanation of Benefits (EoB) that details the costs that the insurer will pay the provider for products and services for the patient. The insurer may send the EoB to the patient so that the patient is aware of any portion of a service or product for which the insurer will pay (e.g., a summary of insurance benefits applied to the claims submitted). The healthcare provider may determine the difference between the costs and the patient's benefits covered by the insurer and may generate a bill to send to the patient to show the patient the amount that the patient owes. However, sometimes an EoB and a provider bill may not match. In addition, the process by which a provider submits claims to providers, and ultimately results in a bill for a patient, may be inefficient and prone to errors and misunderstanding.

Patient healthcare records can be stored as a non-fungible token (NFT) on the blockchain to allow for a “uniqueness” and help prevent duplication of patient healthcare records. By using an NFT storage medium or method, patient healthcare records can be flagged as unique and updated as necessary to include new diagnoses, services, procedures, visits, reporting symptoms, or other data. Each NFT of their healthcare records can be issued to each patient so that the patient can control access to their own healthcare records. An insurance company can issue tokens related to specific procedures instead of issuing benefits. By issuing procedure tokens and using a publicly accessible ledger, the healthcare blockchain system may facilitate identifying tokens that a patient has in their wallet. The billing system here is enhanced, as a healthcare blockchain system may facilitate determining cost, determining the amount of service tokens or value of the service tokens of service or procedure or contact the user, and determine, based on the cost, that the patient owes a certain amount. Removing consideration of insurance policies benefits allows a healthcare provider to more efficiently bill by considering the service or procedure and the value of the patient's wallet.

Therefore, there is a need for an enhanced way of using blockchain and NFTs to facilitate patient billing and manage healthcare records.

Implementation of the NFT' s and storage of the healthcare records can be through the InterPlanetary File System (IPFS), Filecoin, or off-chain storage such as NFT Storage. By using such a system, data can be stored off-chain, preventing the need for large amounts of data to be stored across the blockchain network improving network efficiency and transaction costs.

In one embodiment, an NFT is minted on a choice platform using IPFS that has a current patient's healthcare records stored on it. IPFS created a content identifier (CID) that is directly derived from the data initially added to the NFT. Using the CID, anyone, or any provider, can fetch a copy of the encrypted data from the IPFS network even if the original provider of the records has disappeared. By adding support to the smart contract to update the Universal Resource Indicator (URI) after the token has been issued, NFT's of healthcare records can continuously be updated as more data is added to the record without compromising the uniqueness of their healthcare records, and without further storage of data on-network. Changing the URI to a new IPFS URI still leaves a record of the transaction history to provide accountability, security, and provide a secondary mark of what was changed, when, and by whom, which is not a part of the healthcare record.

In one embodiment, a patient may hold service tokens in a digital wallet. The healthcare blockchain system may facilitate accessing and searching the patient's digital wallet (with user consent and in accordance with relevant laws) using a public key to identify the tokens that patient holds. No direct interaction between the provider and the third party that issued the tokens is necessary. The tokens can be automatically transferred with a smart contract that confirms the service has been administered or goods transferred. The healthcare blockchain system may facilitate an examination to determine if a patient wallet already has the required service tokens to pay for a service or product, and may determine whether a provider may administer the service instead of querying the third party for benefits or waiting for payment from the third party. This benefit is extended to the patient, who does not have to confirm coverage by the third party with the provider before receiving service because the transparency of the wallet, due to the healthcare blockchain system, allows a provider to see the level of coverage a patient can afford. In this manner, the provider may not need to submit a claim to an insurer but instead may determine an amount owed by a patient by accessing the patient's digital wallet and using eligible (e.g., restricted) tokens to pay for products or services rendered. When the patient's digital wallet does not include sufficient token funds for products or services rendered, the healthcare blockchain system may determine the difference between the amount owed by the patient and the amount deducted (or available to be deducted) from the patient's digital wallet as payment and may bill the patient for the difference. However, if the patient's digital wallet does contain sufficient funds to cover the cost for the products or services rendered, the healthcare blockchain system may facilitate subtracting the cost from the patient's digital wallet when authorized by the patient.

The present disclosure has various benefits over current systems, including being a more efficient network for payments and healthcare records. By storing the healthcare records of patients' off-chain, some of the largest data sets do not have to be duplicated for a new user to access or copy the digital blockchain ledger. Instead, only the URI pointing to the healthcare records may be stored on the NFT. This method of off-chain storage means the digital ledger may not have to replicate and store the patient's healthcare data every time a change is made, which can increase the number of transactions per second by an order of magnitude. Off-chain storage also allows for better growth of the network, as the data stored on-chain increases slower since some of the data is stored off-chain. On-chain transactions may refer to transactions available on the blockchain and that are visible to nodes on the blockchain network. Off-chain transactions may include the movement of monetary value outside of the blockchain.

### Illustrative Processes and Use Cases

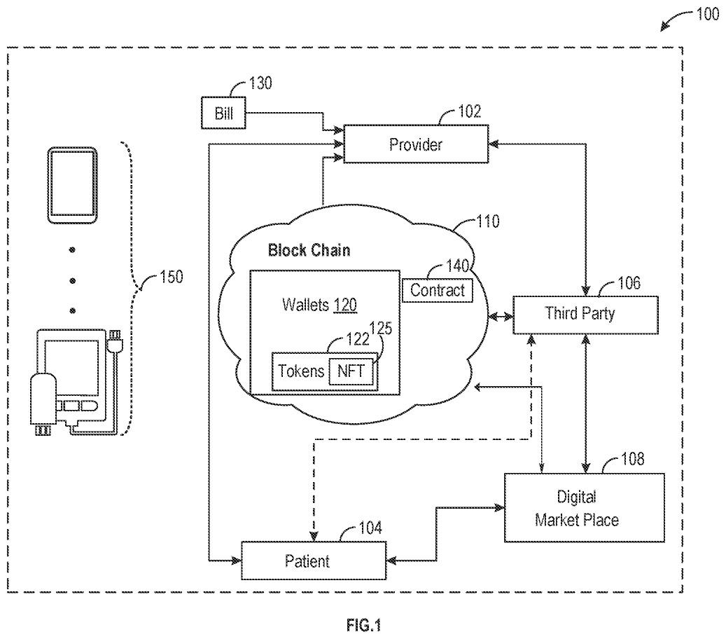

FIG. 1 illustrates an example system 100 for a blockchain ledger 110 available to healthcare providers and patients, in accordance with one or more example embodiments of the present disclosure. The system 100 may comprise a provider group 102, patient group 104, third party group 106, digital marketplace 108, and/or blockchain digital ledger 110. Other components may also be included in the system 100 based on implementation.

Referring to FIG. 1, the system 100 for a blockchain digital ledger 110 is hosted on a blockchain computing system that stores and maintains the blockchain digital ledger 110. The blockchain digital ledger 110 can host smart contracts that use the digital ledger to verify blockchain transactions with the smart contracts. The blockchain digital ledger 110 can comprise both “tokens” and “non-fungible tokens.” The blockchain digital ledger 110 can verify blockchain transactions by comparing the transactions with the smart contract and may transfer tokens accordingly.

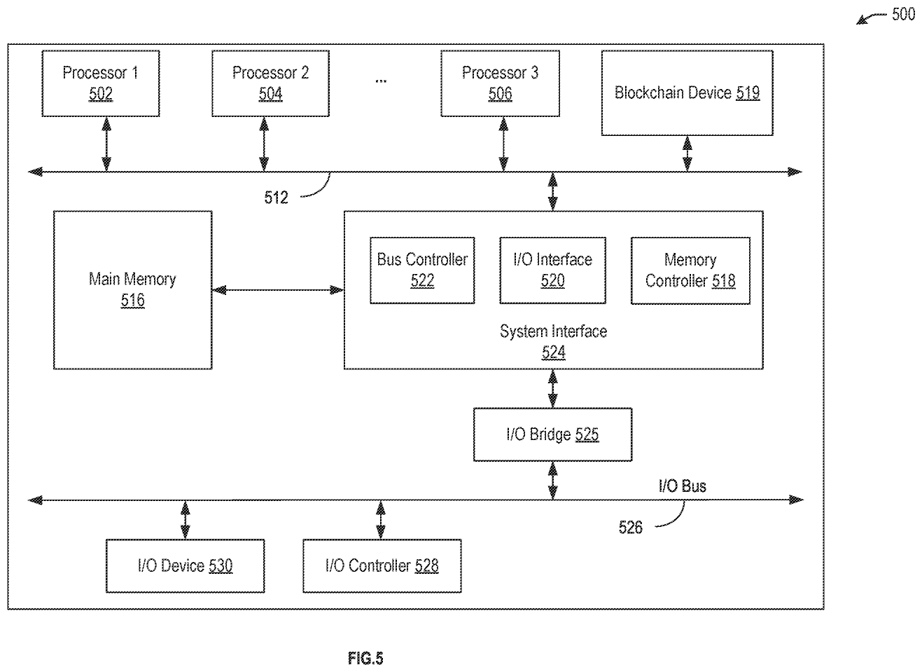

The system 100 for a blockchain digital ledger 110 can have blockchain transactions carried out on it via at least one processor of the healthcare blockchain system (e.g., see FIG. 5). In some embodiments, the at least one processor of the healthcare blockchain system can determine the cost of a healthcare service (e.g., medical, dental, or any other type of healthcare service) provided to a patient by a provider (e.g., a physician, doctor, nurse, etc.). The at least one processor of the healthcare blockchain system can determine this cost by a lookup of stored values corresponding to the services off-chain or on the blockchain digital ledger 110. These services can be medical services, dental services, or other healthcare related services such as services connected to chiropractic care, herbal medicine, or psychology. These services can be tied to any system of categorizing healthcare goods and services such as product codes, Common Procedural Technology (CPT) Codes, or Healthcare Common Procedure Coding System (HCPCS) Codes. The cost can also be determined by the provider entering the cost via the at least one processor of the healthcare blockchain system.

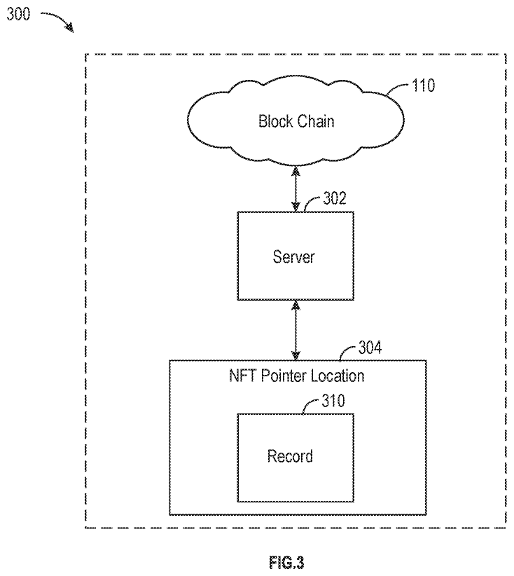

In some embodiments, the at least one processor can access the system 100 for a blockchain digital ledger 110 to determine information. One type of accessible information can be the virtual location and contents of a patient's digital wallet 120. The contents of the patient's digital wallet 120, such as type of tokens 122, number of tokens 122, and restrictions on those tokens 122, can be accessed. In some embodiments, a patient (e.g., of the patient group 104) would have to first give a provider (e.g., of the provider group 102) or other party access to this information via the at least one processor of the healthcare blockchain system. Another type of information accessible by a processor on the blockchain digital ledger 110 is a patient's health record (for example, as shown in FIG. 3). The patient's health record can be stored off-chain, and the non-fungible token (e.g., the record being stored as an NFT 125) may store on-chain a URI pointing to the off-chain storage (e.g., FIG. 3). In other words, accessing the NFT 125 is one step to provide access to the patient's healthcare record by providing its storage location. Multiple patients' healthcare records can be stored together off-chain, or separately off-chain, with differing security, cryptography, or access. This system 100 results in less total data stored on-chain, increasing network efficiency by reducing necessary storage. Off-Chain storage also allows for compliance and flexibility for individual state or country privacy and data storage laws and regulations.

In some embodiments, other types of information that can be stored on-chain, due to their comparatively smaller size of the data compared to the healthcare record, and accessed by the at least one processor of a healthcare blockchain system include banking or financial account information (e.g., a financial account accessible via the blockchain), a patient's second digital wallet information storing other types of tokens or cryptocurrency, benefits information, or information about service providers and pricing.

In some embodiments, instead of using an EoB or information given by an insurance provider or token issuer, the value of the tokens 122 and restrictions are coded. This allows for the at least one processor of the healthcare blockchain system to access a digital wallet of a patient, with patient consent and in accordance with relevant laws, and determine their ability to pay for a service without requiring further information from an insurer. Unlike a process where a patient pays cash, the third-party insurer still is involved in the process via issuing the tokens 122, but is not required to interact in the provider-patient relationship (e.g., to provide the EoB). Thus, this is unlike a situation where a patient pays a provider “out of pocket” with no input from the insurer (e.g., a situation in which no insurance claim is submitted). Instead, the insurer is still involved through their issuance of tokens 122 that allow the healthcare blockchain system to identify how much money the patient has in tokens for a particular service by accessing the patient's digital wallet 120. The present disclosure solves the technical issue of determining if the patient is actually able to pay for the cost of the service without querying the insurer or issuer first to determine how much of a service will be paid for by the insurance provider. The at least one processor of a healthcare blockchain system uses this system 100′s safe and secure access to the patient's digital wallet 120 to determine the patient's ability to pay, and how much. If the patient's digital wallet does contain sufficient funds to cover the cost for the products or services rendered, the healthcare blockchain system may facilitate subtracting the cost from patient's digital wallet, resulting in a difference to be billed to the patient, for example.

In some embodiments, the at least one processor of a healthcare blockchain system can compare the cost of healthcare service to the value of the digital token or tokens in the patient's digital wallet 120, thus determining a difference between the cost and the pecuniary amount provided by the digital token 122. The at least one processor of a healthcare blockchain system can then generate a bill 130 for the patient indicating that the patient owes the difference, or generate a receipt noting that no balance is owed after transferring the digital token 122 to pay for the service rendered.

In one embodiment, the provider group 102 may represent doctors, dentists, hospitals, nurses, administration, and other parties that provide a good or service related to healthcare directly to the patient, and may include devices of the healthcare blockchain system. The provider group 102 and its devices may: (1) access, via at least one processor of a healthcare blockchain system, an encrypted healthcare record associated with a patient who has granted access to the provider group or a subset of a provider group; (2) generate, via at least one processor of a healthcare blockchain system, a request to publish a block to the blockchain digital ledger 110; and (3) view and/or access, via at least one processor of a healthcare blockchain system, the digital wallet 120 of a patient who has granted access to the provider group or a subset of the provider group. The block requested to be generated can include information such as billing information, date, time, information from the encrypted healthcare record accessed, as well as information to update the URI of an NFT 125 to point to an updated healthcare record with the current transaction added.

The provider group 102 and its devices may generate the bill 130 or receipt with the at least one processor after providing a good or service to the patient. These services can be medical services, dental services, or other healthcare related services such as services connected to chiropractic care, herbal medicine, or psychology. These services and goods can be tied to any system of categorizing healthcare goods and services such as product codes, Common Procedural Technology (CPT) Codes, or Healthcare Common Procedure Coding System (HCPCS) Codes.

In some embodiments, the provider group 102 and its devices can prompt the patient group 104 and its devices, via at least one processor of a healthcare blockchain system, to grant access to the patient's encrypted healthcare record, or a portion of their encrypted healthcare record, by publishing a smart contract 140 to the blockchain digital ledger 110. A patient of the patient group 104, using a device, can then accept the request to grant access to the encrypted healthcare record. In some embodiments, the patient group 104 is made up of at least patients and caregivers. The patient group 104 and its devices may: (1) grant access to all or a portion of a patient's encrypted healthcare record ; (2) schedule and receive healthcare goods and services; and (3) pay for received healthcare goods and services. In some embodiments, the third party 106 is an insurer or issuer of the tokens 122 restricted to be exchanged for a particular healthcare service.

In some embodiments, the digital marketplace 108 may allow exchange or lending of the tokens 122 restricted to a good or service. This digital marketplace 108 can be specific to the restricted tokens 122 or can include tokens of other types or other cryptocurrencies. Unlike other digital marketplaces or systems, the system's 100 digital marketplace 108 can facilitate lending and borrowing of tokens as well through the use of liquidity pools or other methods. These liquidity pools use smart contracts to facilitate trading and provide the ability to exchange and, in some embodiments, lend and borrow tokens.

In some embodiments, the patient's digital wallet 120 may be hosted by one or more devices 150 (e.g., representing a “hardware wallet” or a “software wallet”). A hardware or software wallet of the one or more devices 150 may access the blockchain digital ledger 110. For example, a hardware or software wallet of the one or more devices 150 may store a user's private keys to secure storage of the tokens 122. When a patient allows a transaction using the one or more devices 150, the transaction may be signed in the hardware or software wallet, and may be output to the blockchain digital ledger 110 (e.g., through an application on the one or more devices 150).

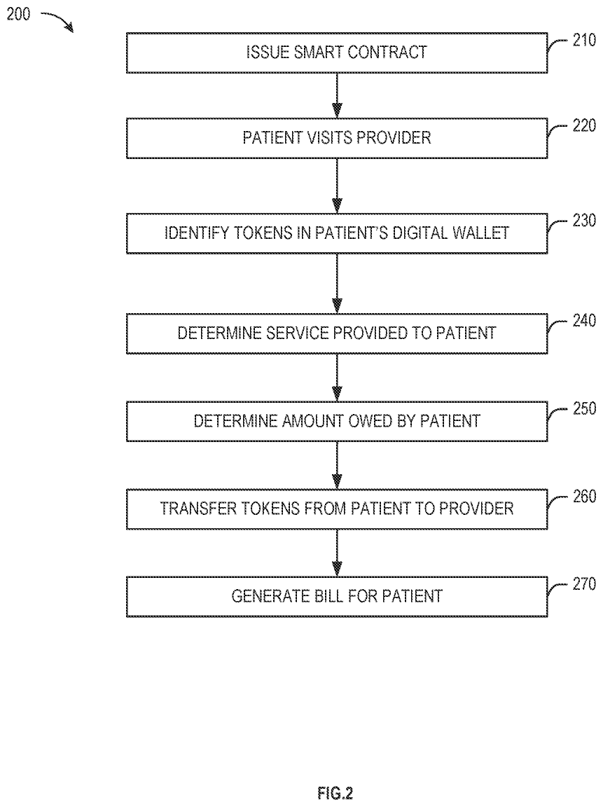

FIG. 2 illustrates an example system flow 200 for using a blockchain digital ledger (e.g., the blockchain digital ledger 110 of FIG. 1) available to healthcare providers and patients, in accordance with one or more example embodiments of the present disclosure. A provider or third party can issue a smart contract (e.g., the smart contract 140 of FIG. 1) at block 210 via at least one processor of a healthcare blockchain system to the digital blockchain ledger 110 to incentivize a patient to receive a healthcare service. The smart contract can be set up to give the patient a token (e.g., the tokens 122 of FIG. 1) or a transfer of pecuniary value based on receiving the good or service. The smart contract can define terms and conditions to receive a specific good or service, receive any good or service from a specific provider, visit a provider or specific provider for a service or product, or other activities that the provider or issuer wishes to incentivize. In this manner, the smart contract may limit a token to use in payment to particular health or dental provider, for a particular service or a particular product.

The patient can visit a provider (e.g., of the provider group 102 of FIG. 1) at block 220, or a specific provider if required by the smart contract, to fulfill the requirements of the smart contract (e.g., for a medical, dental, or other healthcare procedure). The provider can then use the at least one processor (e.g., see FIG. 5) of a provider group device to evaluate the patient wallet for tokens (e.g., see FIG. 1) at block 230 that may be used to pay for the product or procedure provided to the patient. These tokens can be restricted to the specific service that the patient was incentivized to receive, such as a general medical evaluation, a specific operation, etc.

In one example, the patient has 0.75 general medical evaluation tokens in their wallet. The provider provides a service to the patient at block 240, and at least one processor determines the service provided to a patient (e.g., as provided by a user input). Blocks 230 and 240 may be performed in either order. This service can be any healthcare service, such as a general medical evaluation. The provider device then determines the amount owed by the patient at block 250. This is carried out by the at least one processor determining the cost of the service (e.g., based on a user input or based on a lookup value that provides a price corresponding to the service), the value of the tokens in the patient's wallet that are allowed to be used for the service, and determining the difference. The difference in this example would be the amount owed by the patient. In this example, the provider device determines via the at least one processor that the value of a general medical evaluation is $1000. The provider device determines that 1 general medical evaluation token is worth 1 general medical evaluation. The patient only has 0.75 general medical evaluation tokens, so the difference in value is $250 (e.g., 0.75×$1000=$750 in token value, so the amount owed by the patient would be $1000-$750 in token value, or $250). Alternatively, the tokens may provide a specific money value (e.g., $750 for a particular service). The value of the tokens may be based on the patient's insurance policy and its benefits and may be based on how many insurance benefits have been used in comparison to a maximum amount covered by the insurer, a patient deductible, etc. In this manner, rather than the healthcare provider having to contact the insurer to provide the price of services rendered, and relying on the insurer to determine the amount of the price that will be paid by the insurer, the healthcare provider device efficiently and securely may access the digital ledger with the patient's digital wallet to determine, more directly, the amount of money (if any) to be charged to a patient, and to collect at least some of the payment from the digital wallet (e.g., based on the pre-issued tokens made accessible to the healthcare provider for payment). If the tokens provide sufficient monetary value for the services rendered, then the patient may not owe anything, and the healthcare provider may avoid having to bill the patient. However, if the patient's digital wallet does contain sufficient funds to cover the cost for the products or services rendered, the healthcare blockchain system may facilitate subtracting the cost form patient's digital wallet. The values of the services and tokens provided herein are exemplary and not meant to be limiting.

The tokens are transferred from the patient's digital wallet to the provider, via at least one processor of a healthcare blockchain system, at block 260 to the provider's digital wallet or another source when the patient has consented to the healthcare provider receiving tokens that are available for use in paying for services rendered. The patient's digital wallet and provider's digital wallet are stored on the blockchain digital ledger 110 on the system 100. The tokens can be transferred automatically by the smart contract being executed, the provider requesting the tokens be transferred, or the patient transferring the tokens to the healthcare provider. In one embodiment, the smart contract is executed and 0.75 general medical evaluation tokens are transferred from the provider to the patient when a block is updated on the blockchain digital ledger 110 that reflects that the service was provided by the provider to the patient. Another possible payment method is that the smart contract can also transfer the tokens elsewhere, such as a third-party wallet, and cash value can be transferred to the provider from the third party.

A bill (e.g., the bill 130 of FIG. 1) is generated, via at least one processor of a healthcare blockchain system, for the patient at block 270 for the amount that they owe. In this example, the bill would reflect that the patient owes the provider $250 (e.g., the difference between the amount covered by the insurance policy as provided by the tokens and the price of services rendered). The bill can be generated off-chain, or on the digital blockchain ledger 110 as a reflection of the tokens transferred. The at least one processor can access the blockchain digital ledger 110 to ensure the proper amount of tokens was transferred and that the bill is accurate. The bill shows what services were rendered, the price, the amount covered by the insurance (e.g., the token value), and the amount owed by the patient, if any.

FIG. 3 illustrates an example system 300 for a blockchain digital ledger 110 and non-fungible tokens available to healthcare providers and patients, in accordance with one or more example embodiments of the present disclosure. In some embodiments, the non-fungible token 125 can store a pointer to a location 304 off-chain such as a server 302 or a collection of servers where data is stored. In some embodiments, this data includes healthcare record data 310 (e.g., in accordance with relevant laws, such as those related to the privacy and management of personal health information). The at least one processor can determine, via a smart contract (e.g., the smart contract 140 of FIG. 1), where the healthcare record data is stored. A patient can provide access to a provider or third party to all or part of the data via the execution of a smart contract on the blockchain ledger.

For example, the at least one processor can access a smart contract that identifies the pointer for where the healthcare record is stored. The at least one processor can then request access to the location on a server 302 where the healthcare record data is stored. The at least one processor could have already received approval from the patient to access the healthcare data, or the patient could be prompted to grant access now. The at least one processor can the facilitate presentation of the data to confirm or determine the services to render. After the service is rendered, the at least one processor may update the health record 310 at the pointer location 304 of the server 302, and/or the at least one processor may request to generate a block on the blockchain digital ledger 110 to update the pointer to point to an updated healthcare digital record created elsewhere on the server.

The digital health record of the system 300 allows for the patient to control the record by storing the pointer on their NFT. By each patient owning the NFT with their health record, each patient controls access and changes. In addition, this system allows for portability of the health record, as the patient always has access to their health record via the NFT 125. The patient can even allow text or imaging of the record to be included in the NFT 125. In one example, the at least one processor determines that images or notes on services are added to the service record kept by the healthcare provider. The at least one processor can prompt the patient, or the patient can prompt the at least one processor, to add the images and/or text to the NFT 125 or to the NFT pointer location 304. The at least one processor or the system 300 may have access to software that performs the conversion of image data (e.g., from an X-ray) to data stored on an NFT. The data is converted to readable text, compressed and encoded, or stored in any number of ways to improve the efficiency of the network. This automatic conversion of imaging data prevents future providers from having to request this data from someone other than the patient, keeping the patient in control of their entire health record, improving cost of provider storage, portability, and efficiency of the interaction between future providers and the patient.

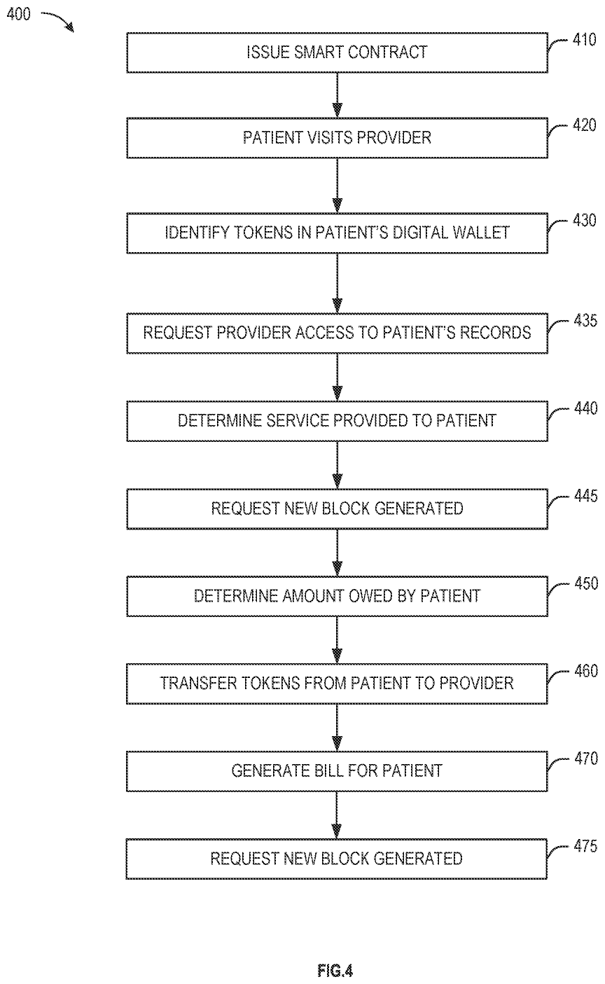

FIG. 4 illustrates an example process flow 400 for using a blockchain ledger available to healthcare providers and patients, in accordance with one or more example embodiments of the present disclosure. A provider or third party can issue a smart contract at block 410 via at least one processor of a healthcare blockchain system to the digital blockchain ledger to incentivize a patient to receive a healthcare service. The smart contract can be set up to give the patient a token or a transfer of pecuniary value based on receiving the good or service. The smart contract can be to receive a specific good or service, receive any good or service from a specific provider, simply to go to a provider or specific provider, or other activities that the provider or issuer wishes to incentivize.

The patient can visit a provider at block 420 or a specific provider if required by the smart contract to fulfill the requirements of the smart contract. The provider can then use the at least one processor of a healthcare blockchain system to check the patient wallet for tokens at block 430. These tokens can be restricted to the specific service that the patient was incentivized to receive, such as a general medical evaluation. In one example, the patient has 0.75 general medical evaluation tokens in their digital wallet.

The provider (e.g., a device of the provider group 102 of FIG. 1) can then request access to the patient's health records, via at least one processor of a healthcare blockchain system, at block 435 (e.g., as stored using an NFT). The patient can confirm access of all or part of the patient's health records. The provider's device can access the health records in an off-chain storage system. “Access” in this context is the ability to read the unencrypted version of the patient's health record. In this example, the health records are stored off-chain in a server. The patient's wallet contains a non-fungible token that has a URI for the location of the health records stored on it. The health records may be encrypted such that, even with the URI, the patient still has to give access to the health records, or the URI itself can be encrypted with the patient giving access to the URI and thus access to the health records. After the patient provides the provider device access, the provider device can then access all or part of the patient's health records via at least one processor of a healthcare blockchain system. The provider provides a service to the patient at block 440, and the healthcare blockchain system may determine what service was provided to the patient (e.g., via a user input). Block 440 may occur before or after block 430. The provided service can be any healthcare service, such as a general medical evaluation.

The provider device requests a new block to be generated at block 445 on the blockchain digital ledger via at least one processor of a healthcare blockchain system. This new block can show that the provider provided the service or update the URI to reflect an updated copy of the patient's health records showing that the patient received the service. The information on the block can also show that the patient has or has not yet paid for the service provided.

The provider device determines the amount owed by the patient at block 450. This is carried out by the at least one processor of a healthcare blockchain system determining the service, the cost of the service, the value of the tokens in the patient's wallet, and determining the difference. The service can be determined by input from the provider, accessing the provider's system to see the service provided, accessing the digital blockchain ledger to see the service provided on the ledger, or accessing the updated patient's health record. The difference here would be the amount owed. In the example started above, the provider determines via the at least one processor that the value of a general medical evaluation is $1000. The provider determines that 1 general medical evaluation token is worth 1 general medical evaluation. The patient only has 0.75 general medical evaluation tokens, so the difference in value is $250.

The tokens are transferred from the patient to the provider at block 460 via at least one processor of a healthcare blockchain system. The tokens can be transferred automatically by the smart contract being executed, the provider requesting the tokens be transferred, or the patient transferring the tokens. In one embodiment, the smart contract is executed and 0.75 general medical evaluation tokens are transferred from the provider to the patient when a block is updated on the blockchain digital ledger that reflects that the service was provided by the provider to the patient.

A bill is generated by a device of the healthcare blockchain system for the patient at block 470 for the amount that they owe. In this example, the bill would reflect that the patient owes the provider a further $250. The bill can be generated off-chain, or on the digital blockchain ledger as a reflection of the tokens transferred. The at least one processor can access the blockchain digital ledger to ensure the proper amount of tokens was transferred and that the bill is accurate.

The provider device requests a new block to be generated at block 475 via at least one processor of a healthcare blockchain system on the blockchain digital ledger if necessary. This new block can show that the provider provided the service or update the URI to reflect an updated copy of the patient's health records showing that the patient received the service. The information on the block can also show that the patient has or has not yet paid, in part or in full, for the service provided, or that the bill has been generated or not.

Referring to FIGS. 2 and 4, any one or more devices of the healthcare blockchain system (e.g., the system 100 of FIG. 1) may perform any steps of the processes 200 and 400 (excluding blocks 220 and 420, which represent the patient visiting a provider). The one or more devices of the healthcare blockchain system used to perform steps of the processes 200 and 400 may include devices of the provider group 102, devices of the patient group 104, the one or more devices 150, etc.

It is understood that the above descriptions are for purposes of illustration and are not meant to be limiting.

FIG. 5 is a block diagram illustrating an example of a computing device or computer system 500 upon which any of one or more techniques (e.g., methods) may be performed, in accordance with one or more example embodiments of the present disclosure.

For example, the computing system 500 of FIG. 5 may represent a portion of or include a portion of the systems in FIGS. 1 and 3, and may include components as described herein to facilitate the processes described in FIGS. 1-4. In this manner, devices of the provider group 102, devices of the patient group 104, devices of the third party group 106, and/or the one or more devices 150 of FIG. 1 may include at least some of the components shown in FIG. 5. The computer system (system) includes one or more processors 502-506. Processors 502-506 may include one or more internal levels of cache (not shown) and a bus controller (e.g., bus controller 522) or bus interface (e.g., I/O interface 520) unit to direct interaction with the processor bus 512. A healthcare blockchain device 519 may include one or more modules capable of performing any of the steps described in FIGS. 1-4, may also be in communication with the processors 502-506, and may be connected to the processor bus 512, allowing for analysis of payment and insurance information from the perspective of a health or dental provider, an insurance provider, or a patient.

Processor bus 512, also known as the host bus or the front side bus, may be used to couple the processors 502-506 and/or the blockchain device 519 with the system interface 524. System interface 524 may be connected to the processor bus 512 to interface other components of the system 500 with the processor bus 512. For example, system interface 524 may include a memory controller 518 for interfacing a main memory 516 with the processor bus 512. The main memory 516 typically includes one or more memory cards and a control circuit (not shown). System interface 524 may also include an input/output (I/O) interface 520 to interface one or more I/O bridges 525 or I/O devices 530 with the processor bus 512. One or more I/O controllers and/or I/O devices may be connected with the I/O bus 526, such as I/O controller 528 and I/O device 530, as illustrated.

I/O device 530 may also include an input device (not shown), such as an alphanumeric input device, including alphanumeric and other keys for communicating information and/or command selections to the processors 502-506 and/or the blockchain device 519. Another type of user input device includes cursor control, such as a mouse, a trackball, or cursor direction keys for communicating direction information and command selections to the processors 502-506 and/or the blockchain device 519 and for controlling cursor movement on the display device.

System 500 may include a dynamic storage device, referred to as main memory 516, or a random access memory (RAM) or other computer-readable devices coupled to the processor bus 512 for storing information and instructions to be executed by the processors 502-506 and/or the blockchain device 519. Main memory 516 also may be used for storing temporary variables or other intermediate information during the execution of instructions by the processors 502-506 and/or the blockchain device 519. System 500 may include read-only memory (ROM) and/or other static storage device coupled to the processor bus 512 for storing static information and instructions for the processors 502-506 and/or the blockchain device 519. The system outlined in FIG. 5 is but one possible example of a computer system that may employ or be configured in accordance with aspects of the present disclosure.

According to one embodiment, the above techniques may be performed by computer system 500 in response to processor 504 executing one or more sequences of one or more instructions contained in main memory 516. These instructions may be read into main memory 516 from another machine-readable medium, such as a storage device. Execution of the sequences of instructions contained in main memory 516 may cause processors 502-506 and/or the blockchain device 519 to perform the process steps described herein. In alternative embodiments, circuitry may be used in place of or in combination with the software instructions. Thus, embodiments of the present disclosure may include both hardware and software components.

Various embodiments may be implemented fully or partially in software and/or firmware. This software and/or firmware may take the form of instructions contained in or on a non-transitory computer-readable storage medium. Those instructions may then be read and executed by one or more processors to enable the performance of the operations described herein. The instructions may be in any suitable form, such as, but not limited to, source code, compiled code, interpreted code, executable code, static code, dynamic code, and the like. Such a computer-readable medium may include any tangible non-transitory medium for storing information in a form readable by one or more computers, such as but not limited to read-only memory (ROM); random access memory (RAM); magnetic disk storage media; optical storage media; a flash memory, etc.

A machine-readable medium includes any mechanism for storing or transmitting information in a form (e.g., software, processing application) readable by a machine (e.g., a computer). Such media may take the form of, but is not limited to, non-volatile media and volatile media and may include removable data storage media, non-removable data storage media, and/or external storage devices made available via a wired or wireless network architecture with such computer program products, including one or more database management products, web server products, application server products, and/or other additional software components. Examples of removable data storage media include Compact Disc Read-Only Memory (CD-ROM), Digital Versatile Disc Read-Only Memory (DVD-ROM), magneto-optical disks, flash drives, and the like. Examples of non-removable data storage media include internal magnetic hard disks, solid state devices (SSDs), and the like. The one or more memory devices (not shown) may include volatile memory (e.g., dynamic random access memory (DRAM), static random access memory (SRAM), etc.) and/or non-volatile memory (e.g., read-only memory (ROM), flash memory, etc.).

Computer program products containing mechanisms to effectuate the systems and methods in accordance with the presently described technology may reside in main memory 516, which may be referred to as machine-readable media. It will be appreciated that machine-readable media may include any tangible non-transitory medium that is capable of storing or encoding instructions to perform any one or more of the operations of the present disclosure for execution by a machine or that is capable of storing or encoding data structures and/or modules utilized by or associated with such instructions. Machine-readable media may include a single medium or multiple media (e.g., a centralized or distributed database, and/or associated caches and servers) that store the one or more executable instructions or data structures.

Embodiments of the present disclosure include various steps, which are described in this specification. The steps may be performed by hardware components or may be embodied in machine-executable instructions, which may be used to cause a general-purpose or special-purpose processor programmed with the instructions to perform the steps. Alternatively, the steps may be performed by a combination of hardware, software, and/or firmware.

Various modifications and additions can be made to the exemplary embodiments discussed without departing from the scope of the present disclosure. For example, while the embodiments described above refer to particular features, the scope of this disclosure also includes embodiments having different combinations of features and embodiments that do not include all of the described features. Accordingly, the scope of the present disclosure is intended to embrace all such alternatives, modifications, and variations together with all equivalents thereof.

The operations and processes described and shown above may be carried out or performed in any suitable order as desired in various implementations. Additionally, in certain implementations, at least a portion of the operations may be carried out in parallel. Furthermore, in certain implementations, less than or more than the operations described may be performed.

The word “exemplary” is used herein to mean “serving as an example, instance, or illustration.” Any embodiment described herein as “exemplary” is not necessarily to be construed as preferred or advantageous over other embodiments.

As used herein, unless otherwise specified, the use of the ordinal adjectives “first,” “second,” “third,” etc., to describe a common object, merely indicates that different instances of like objects are being referred to and are not intended to imply that the objects so described must be in a given sequence, either temporally, spatially, in ranking, or any other manner.

It is understood that the above descriptions are for purposes of illustration and are not meant to be limiting.

Although specific embodiments of the disclosure have been described, one of ordinary skill in the art will recognize that numerous other modifications and alternative embodiments are within the scope of the disclosure. For example, any of the functionality and/or processing capabilities described with respect to a particular device or component may be performed by any other device or component. Further, while various illustrative implementations and architectures have been described in accordance with embodiments of the disclosure, one of ordinary skill in the art will appreciate that numerous other modifications to the illustrative implementations and architectures described herein are also within the scope of this disclosure.

Although embodiments have been described in language specific to structural features and/or methodological acts, it is to be understood that the disclosure is not necessarily limited to the specific features or acts described. Rather, the specific features and acts are disclosed as illustrative forms of implementing the embodiments. Conditional language, such as, among others, “can,” “could,” “might,” or “may,” unless specifically stated otherwise, or otherwise understood within the context as used, is generally intended to convey that certain embodiments could include, while other embodiments do not include, certain features, elements, and/or steps. Thus, such conditional language is not generally intended to imply that features, elements, and/or steps are in any way required for one or more embodiments or that one or more embodiments necessarily include logic for deciding, with or without user input or prompting, whether these features, elements, and/or steps are included or are to be performed in any particular embodiment.

1. A method for secure blockchain transactions, the method comprising:
determining, by at least one processor of a device, a cost of a healthcare service provided to a patient by a healthcare provider;
accessing, by the at least one processor, a blockchain digital ledger;
identifying, by the at least one processor, a digital wallet of the patient stored using the blockchain digital ledger;
identifying, by the at least one processor, a digital token stored in the digital wallet, the digital token restricted to payment for the healthcare service, and the digital token issued by an insurer;
determining, by the at least one processor, a difference between the cost and a pecuniary amount provided by the digital token; and
generating, by the at least one processor, a bill for the patient, the bill indicating that the patient owes the difference to the medical or dental provider.

2. The method of claim 1, further comprising:
accessing a non-fungible token of the blockchain digital ledger, wherein the non-fungible token comprises a healthcare record of the patient.

3. The method of claim 2, wherein the non-fungible token comprises a uniform resource identifier identifying a location where the healthcare record of the patient is stored off the blockchain digital ledger.

4. The method of claim 2, wherein accessing the non-fungible token comprises:
requesting, by the at least one processor, access from the patient to the healthcare record of the patient; and
receiving, by the at least one processor, access granted by the patient to the healthcare record of the patient.

5. The method of claim 1, further comprising:
identifying, by the at least one processor, a financial account of the patient; and
transferring, based on a smart contract of the blockchain digital ledger, the difference between the cost and the pecuniary amount provided by the digital token from a financial account of the patient to a financial account of the medical or dental provider.

6. The method of claim 1, further comprising:
identifying, by the at least one processor, a second digital wallet of the patient stored using the blockchain digital ledger
transferring, based on a smart contract of the blockchain digital ledger, the difference between the cost and the pecuniary amount provided by the digital token from a second digital wallet of the patient to the digital wallet of the healthcare provider.

7. The method of claim 1, further comprising:
generating, by the least one processor, a smart contract including a token incentive for the patient to receive a specific healthcare service.

8. The method of claim 1, further comprising:
issuing, based on a smart contract of the blockchain digital ledger, the digital token by an insurer.

9. The method of claim 1, further comprising transferring, based on a smart contract of the blockchain digital ledger, the digital token from the first digital wallet of the patient to the medical or dental provider.

10. The method of claim 1, wherein identifying a digital wallet of the patient is based on a smart contract of the blockchain digital ledger.

11. The method of claim 1, further comprising requesting, by the at least one processor, generation of a new block on the blockchain digital ledger, the new block indicating that the healthcare service was provided to the patient.

12. The method of claim 1, wherein the token is configured to be exchanged or lent by patients on a digital marketplace.

13. A system for secure healthcare digital transactions, comprising:
a blockchain digital ledger;
a digital wallet, configured to store a digital token; and
at least one processor, configured to:
determine a cost of a healthcare service provided to a patient by a healthcare provider;
access the blockchain digital ledger;
identify the digital wallet of the patient stored using the blockchain digital ledger;
identify the digital token stored in the digital wallet, the digital token restricted to payment for the healthcare service, and the digital token issued by an insurer;
determine a difference between the cost and a pecuniary amount provided by the digital token; and
generate a bill for the patient, the bill indicating that the patient owes the difference to the medical or dental provider.

14. The system of claim 13, wherein the at least one processor is further configured to:
access a non-fungible token of the blockchain digital ledger, wherein the non-fungible token comprises a healthcare record of the patient.

15. The system of claim 14, wherein the non-fungible token comprises a uniform resource identifier identifying a location where the healthcare record of the patient is stored off the blockchain digital ledger.

16. The system of claim 13, wherein to access the non-fungible token comprises the at least one processor being further configured to:
request access from the patient to the healthcare record of the patient; and
receive access granted by the patient to the healthcare record of the patient.

17. The system of claim 13, wherein the at least one processor is further configured to:
generate a smart contract including a token incentive for the patient to receive a specific healthcare service.

18. A non-transitory computer-readable medium storing computer-executable instructions which when executed by one or more processors result in performing operations comprising:
determining a cost of a healthcare service provided to a patient by a healthcare provider;
accessing a blockchain digital ledger;
identifying a digital wallet of the patient stored using the blockchain digital ledger;
identifying a digital token stored in the digital wallet, the digital token restricted to payment for the healthcare service, and the digital token issued by an insurer;
determining a difference between the cost and a pecuniary amount provided by the digital token; and
generating a bill for the patient, the bill indicating that the patient owes the difference to the medical or dental provider.

19. The non-transitory computer-readable medium of claim 18, the operations further comprising:
accessing a non-fungible token of the blockchain digital ledger, wherein the non-fungible token comprises a healthcare record of the patient.

20. The non-transitory computer-readable medium of claim 19, wherein the non-fungible token comprises a uniform resource identifier identifying a location where the healthcare record of the patient is stored off the blockchain digital ledger.
